(19) United States
(12) Patent Application Publication
(10) Pub. No.: US 2007/0029363 A1
(43) Pub. Date: Feb. 8, 2007

(54) Surgical apparatus with remote drive

(76) Inventor: Sergey Popov, Beer Sheva (IL)

Correspondence Address: DANIEL J SWIRSKY, 55 REUVEN ST., BEIT SHEMESH, 99544, IL

(21) Appl. No.: 11/499,856

(22) Filed: Aug. 7, 2006

(30) Foreign Application Priority Data
Aug. 7, 2005 (IL) 170,134

(51) Int. Cl. A61B 17/10

(52) U.S. Cl. 227/175.1

(57) ABSTRACT

A surgical apparatus, having a surgical assembly, a power unit providing the needed power for actuating said surgical assembly, a drive assembly connecting said power unit and said surgical assembly, being flexible at least in the limits of the part of its length, and including an elongated body, and at least of one drive member designed for transmitting a driving motion from said power unit to said surgical assembly and movably disposed within said elongated body, a distal coupling means for coupling the distal ends of said drive assembly to said surgical assembly, a separable proximal coupling means for detachable coupling the proximal ends of said drive assembly to said power unit, an operating means designed for an operation control of said surgical assembly and disposed on said drive assembly distally of said separable proximal coupling means.

### BACKGROUND OF THE INVENTION

1. Field of the Invention

The present invention relates to the surgical instruments with a remote actuation. Specifically, it relates to the surgical instruments with the remote electromechanical drive, flexible drive shaft, and the means of remote operation control.

2. Prior Art

The surgical apparatus with anastomosing, stapling, or resecting executive head remotely powered by power unit, like an electric motor, through flexible drive shaft provides the certain advantages. Specifically, the apparatus actuation and the operation control of the power unit are convenient since do not demand the considerable efforts of surgeon. At the same time, the flexible drive shaft hampers holding the apparatus and emplacement of its head. Moreover, there are some problems of the surgical head control and sterilizing the apparatus reusable parts.

U.S. Pat. No. 6,443,973 discloses an electromechanical surgical device for use in gastrointestinal tract surgery. The device includes an electromechanical driver with at least one electric motor housed in the apparatus handle and the flexible shaft transmitting the motor torque to a surgical head coupled to the distal end of the flexible shaft. The flexible shaft is inseparably coupled to the apparatus handle and detachably coupled to the surgical head. The handle is provided with the means for the motor control as well as the steering wheel connected by the steering wires to the flexible shaft distal end to steer the surgical head position. The flexible shaft and the handle with the electric motor are the reusable components of the surgical device. The disadvantage of the device is the considerable distance between the apparatus handle and surgical head (approximately 2 m), which along with the shaft flexibility hamper the surgical head emplacement with needed accuracy. Another disadvantage is the large length of the steering wires passing through the all length of the flexible shaft complicating the apparatus design, heightening its cost, as well as inevitably acting on the flexible shaft function properties. Moreover, the inseparable coupling of the flexible shaft to the handle leads to the necessity of sterilizing also the handle with the electric motor and control means, which are disposed at the sufficient distance from the operation field and do not subject to contamination. It should be noted, that subsequent attempts to avoid the sterilization of the electric motor and control means by using the detachable coupling the flexible shaft to the electric motor have led to eliminating the mentioned means for steering the surgical head since carrying out the detachable coupling of steering wires to the existing control means is very problematical.

### SUMMARY OF THE INVENTION

An objective of the present invention is improving the convenience of the apparatus operation control by surgeon.

Another objective is heightening the accuracy of the surgical head emplacement.

Another objective is avoiding the sterilization of the power unit without eliminating the means for steering the surgical head.

Another objective is simplifying the design and reducing the cost of the mechanism for steering the surgical head.

Another objective is providing the easy adaptability of the operation control means to the various surgical situations.

The above noted objectives are accomplished with a surgical apparatus having a surgical assembly, a power unit providing the needed power for actuating the surgical assembly, and a drive assembly connecting the power unit and surgical assembly. The drive assembly is flexible at least in the limits of the part of its length and includes an elongated body, and at least of one drive member. The latter is designed for transmitting a driving motion from the power unit to the surgical assembly and movably disposed within the elongated body. There are also a distal coupling means for coupling the distal end of the drive assembly to the surgical assembly, and a separable proximal coupling means for detachable coupling the proximal end of the drive assembly to the power unit. The apparatus is provided with an operating means designed for an operation control of the surgical assembly and disposed on the drive assembly distally of the separable proximal coupling means. The operating means has a stiffening means including a rigid element participating in the transmission of surgeon controlling efforts directed to the surgical assembly. The rigid element provides substantial easing the surgical assembly emplacement and heightening the emplacement accuracy. In version embodiment, the rigid element is made as a rigid portion of the drive assembly disposed between two flexible portions of the drive assembly, therewith the flexible and rigid portions are inseparable. In this design, the distal flexible portion of the drive assembly is designed for steering the surgical assembly.

In another version embodiment, the drive assembly consists of a distal segment and a proximal segment detachable from one another, the distal segment is inseparably coupled to the surgical assembly, and the operating means is disposed on the distal segment.

In another version embodiment, the drive assembly is made as an inseparable unit consisting of the rigid and flexible end portions, the distal and proximal coupling means are separable, and both coupling means have identical design allowing the use of any end of the drive assembly for coupling to the power unit and correspondently to the surgical assembly. This provides the easy adaptability of the surgical apparatus, allowing coupling the surgical assembly to rigid or flexible end of the drive assembly depending on the needs of surgical situation.

In version embodiment, the operation means is detachably mounted on the drive assembly by a mounting means. The mounting means includes a resilient member like a rubber spacer compressed between a clamping nut and a base of the operating means and forced out in the direction of the drive assembly thereby fixing the operating means on the drive assembly. Specifically, the stiffening means is made as a supporting means having the rigid element detachably set on the flexible portion of the drive assembly by the mentioned mounting means to impart the rigidity to the flexible portion of drive assembly during the surgical apparatus operation.

The operating means is provided with a holding means including a handle disposed on the drive assembly and designed for holding the surgical apparatus by surgeon during the execution of the surgical operations. The holding means is mounted on the drive assembly and specifically made as a part of the detachable supporting means. There is also an operation desk having the wire or wireless communication with the power unit, disposed on the holding means, and designed for the surgeon control of the surgical assembly motions including at least the control of the power unit.

The surgical apparatus with the flexible distal end portion of the drive assembly is provided with a steering means with at least one steering wire disposed within the drive assembly along its axis and connecting the flexible distal end portion of drive assembly with a steering member disposed on the holding means distally of the proximal separable coupling means. Thus, the steering wires pass only through a relatively short portion of the drive assembly between the holding means and the flexible distal end portion of drive assembly. This allows reducing the steering means cost. Moreover, the inseparability of the steering means heightens its reliability and durability and simplifies the washing and sterilization of the apparatus.

### BRIEF DESCRIPTION OF THE DRAWINGS

FIG. 1 shows the inseparable drive assembly consisting of the distal end rigid segment and the proximal end flexible segment provided with the identical distal and proximal separable coupling means.

FIG. 2 shows the inseparable drive assembly including a rigid portion disposed between two flexible end portions provided with the identical distal and proximal separable coupling means.

FIG. 3 shows the surgical apparatus having a flexible drive assembly with separable coupling means connecting a stapling head to an electric motor, and a handle disposed along the axis of the drive assembly, fixed on the drive assembly by a mounting means and including an operation desk.

FIGS. 4, 5 show the top and side views respectively of the handle and operation desk of the surgical apparatus shown in FIG. 3.

FIG. 6 shows the handle detachably mounted on the distal rigid end segment of the drive assembly at right angle to the axis of the drive assembly.

FIG. 7 shows the handle inseparably mounted on the rigid distal end segment of the drive assembly and having detachable coupling to the surgical assembly and to the proximal segment (not shown) of the drive assembly.

FIG. 8 shows the handle made as a part of the detachable supporting means including the curvilinear rigid element mounted on the flexible drive assembly.

FIG. 9 shows the drive assembly with an intermediate rigid portion on which the holding means is mounted including the handle and the steering member connected with the flexible distal end portion of the drive assembly by two steering wires.

FIG. 10 shows the cross-sectional view of the drive assembly with disposed therein the steering wires and drive members.

### DETAILED DESCRIPTION OF THE INVENTION

The detailed description of the invention is offered with the references made to the attached drawings in FIGS. 1 to 10.

FIG. 1 shows the inseparable drive assembly 11 consisting of elongated body 12 and flexible drive member 13 designed for transmitting a driving motion from the power unit to the surgical assembly and movably disposed within elongated body 12. The latter includes distal rigid end segment 14 inseparably connected to proximal flexible end segment 15. Drive assembly 11 is provided with distal separable coupling means 16 designed for detachable coupling drive assembly 11 to the surgical assembly and including coupling elements 17 and 18 located on the distal ends of drive member 13 and elongated body segment 14 respectively. There is also proximal separable coupling means 19 designed for detachable coupling drive assembly 11 to the power unit including coupling elements 20 and 21 located on the proximal ends of drive member 13 and elongated body segment 15 respectively. The distal and proximal coupling means have identical design of the collet type for the drive member and of the thread type for the elongated body allowing the use of any end of drive assembly 11 for coupling to the power unit and correspondently to the surgical assembly. Evidently, that any another types of the coupling means, including for example a bayonet type, can be applied.

Distal rigid end segment 14 made as a rigid portion of drive assembly 11 is a rigid element of a stiffening means participating in the transmission of surgeon controlling efforts directed to the surgical assembly. Segment 14 stiffens the flexible drive assembly thereby improving the controllability of the surgical apparatus. Therefore, segment 14 and the stiffening means as a whole are the constituents of an operating means.

In version embodiment, distal segment 14 and proximal segment 15 are coupled by an intermediate separable coupling means allowing their detachment from one another and in principle shown in FIG. 7.

In another version embodiment (not shown), the distal segment of the drive assembly is inseparably coupled to the surgical assembly.

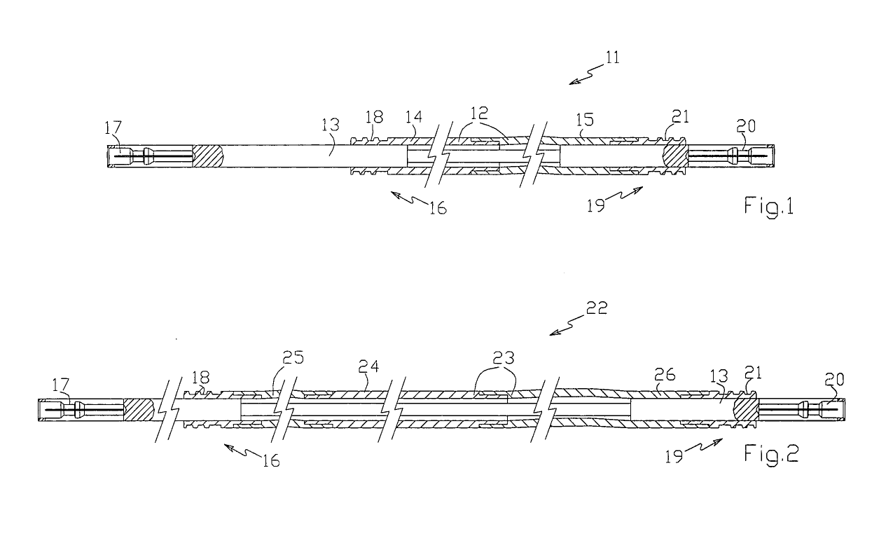

FIG. 2 shows the inseparable drive assembly 22, which, as distinct from drive assembly 11, has elongated body 23 including intermediate rigid portion 24 located between proximal flexible end portion 26 and relatively short distal flexible end portion 25. The latter is designed for steering the surgical assembly. Otherwise, drive assemblies 11 and 22 are identical.

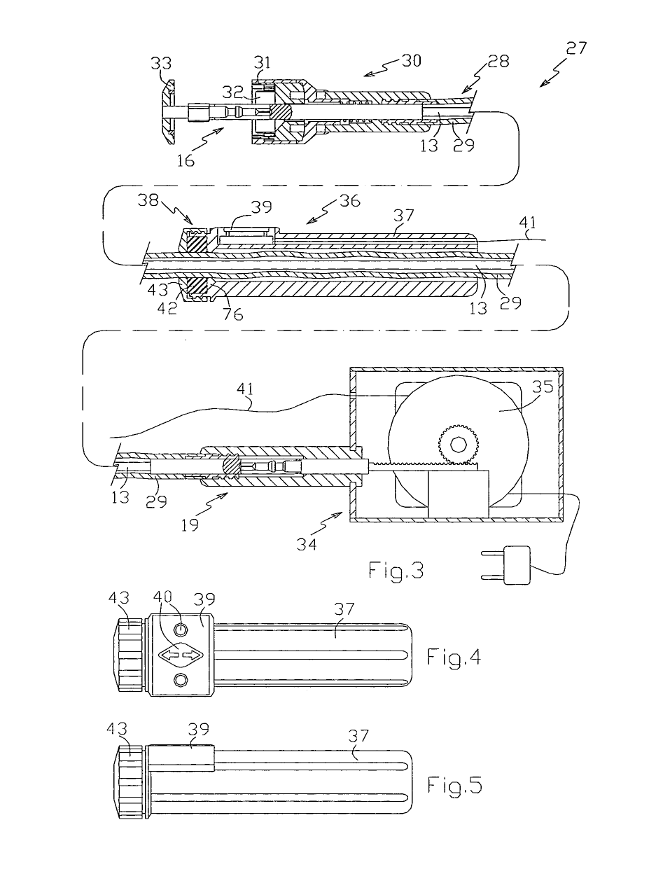

FIGS. 3 to 5 show surgical apparatus 27 having completely flexible drive assembly 28 including flexible elongated body 29 and pushed/pulled flexible drive member 13. Drive assembly 28 has separable coupling means 16 and 19 coupling drive assembly 28 to the surgical assembly 30 and to power unit 34 with electric motor 35 respectively. Surgical assembly 30 is made as a stapling head including staple cartridge 31, knife 32 and anvil 33. The operating means of surgical apparatus 27 is provided with detachable holding means 36 including base 76 and handle 37 designed for holding surgical apparatus 27 by surgeon during the execution of the surgical operations. Handle 37 is disposed along the axis of drive assembly 28 substantially parallel with it, detachably fixed on the drive assembly by mounting means 38, and includes operation desk 39 with controlling members 40 for the control of power unit 34. Operation desk 39 is inseparably mounted on handle 37 and has wire communication 41 with electric motor 35 for transmitting the control signals from the operation desk to the power unit. In version embodiment (not shown), the operation desk is detachably mounted on the handle. In another version embodiment (not shown), the operation desk has wireless communication with the power unit. Mounting means 38 includes resilient rubber spacer 42 compressed between clamping nut 43 and base 76 and forced out in the direction of driving assembly 28. Handle 37 is very convenient specifically for the surgical operations on rectum. Detachable holding means 36 allows surgeon to set handle 37 and operation desk 39 at the needed distance from surgical assembly 30 thereby improving the operation convenience and the apparatus controllability. The same advantages are provided also with the disposition of operation desk 39 on handle 37. Moreover, the latter provides the capabilities of one-handed holding and control of the surgical apparatus during the surgical operations. The presence of proximal separable coupling means 19 and the disposition of holding means 36 and controlling means 39 distally of coupling means 19 create the favorable conditions for the sterilization of the apparatus reusable component, specifically allow avoiding the sterilization of power unit 34.

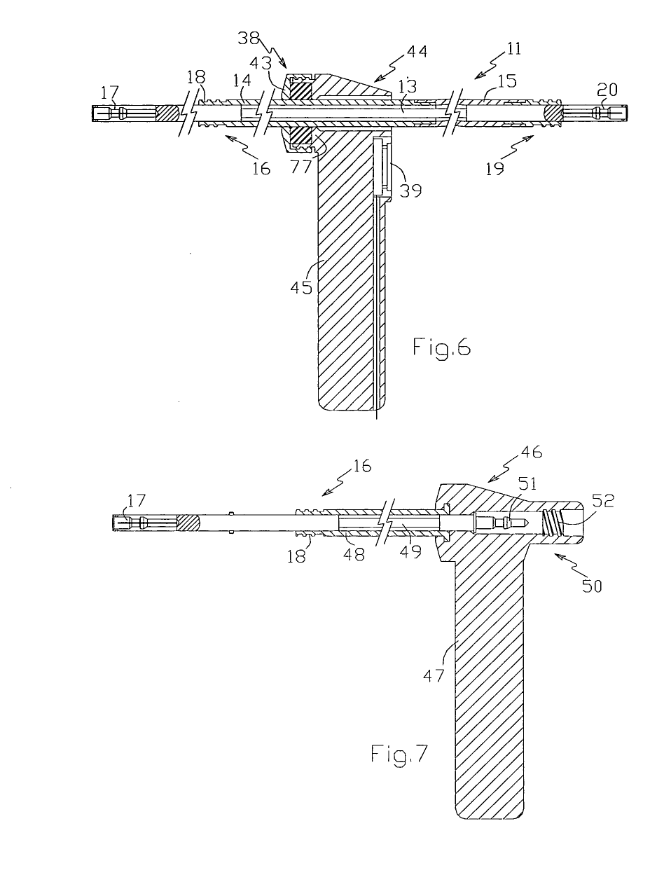

FIG. 6 shows holding means 44 including base 77 and handle 45 detachably mounted on distal rigid end segment 14 of drive assembly 11 at right angle to the axis of the drive assembly. Distal rigid end segment 14 is identical to shown in FIG. 1. Holding means 44 includes mounting means 38 and operation desk 39 identical to shown in FIG. 3. Handle 45 is the most suitable for the surgical operations with using trocars.

FIG. 7 shows holding means 46 including handle 47 inseparably mounted at right angle on rigid distal end segment 48 of the drive assembly. This segment includes flexible drive member 49 and is provided with separable coupling means 16 for detachable coupling to the surgical assembly and separable coupling means 50 with its constituents 51, 52 for detachable coupling to the proximal segment (not shown) of the drive assembly.

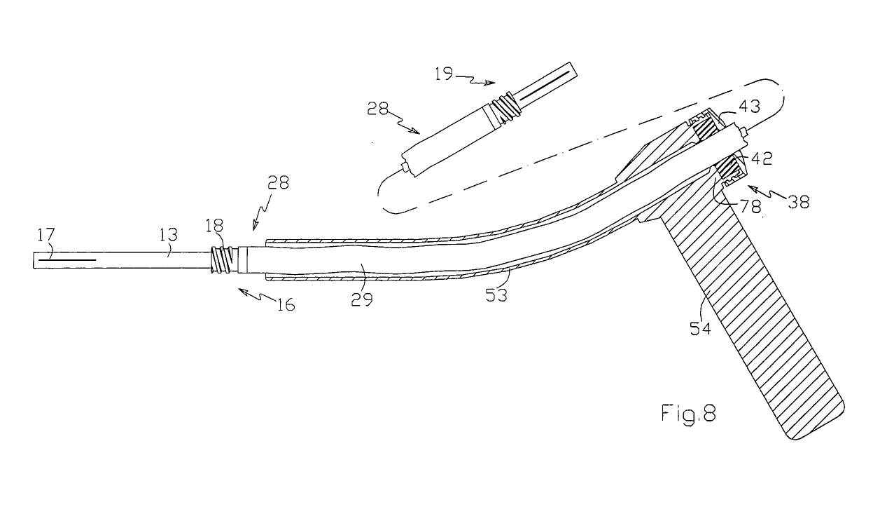

FIG. 8 shows the stiffening means made as a detachable supporting means having base 78 and curvilinear rigid element 53 detachably set on flexible drive assembly 28 by mounting means 38 to impart a rigidity to a portion of flexible drive assembly 28 during the surgical apparatus operation. Therewith, the holding means in the form of handle 54 is made as a part of the detachable supporting means. Mounting means 38 is identical to shown in FIG. 3. The design of the shown detachable supporting means and mounting means allows the emplacement and removal of the supporting means by its axial movement along the drive assembly. In version embodiment (not shown), the design of the detachable supporting means and the mounting means is adapted for the emplacement and removal of the supporting means from the side of the drive assembly by its side movement with respect to the drive assembly.

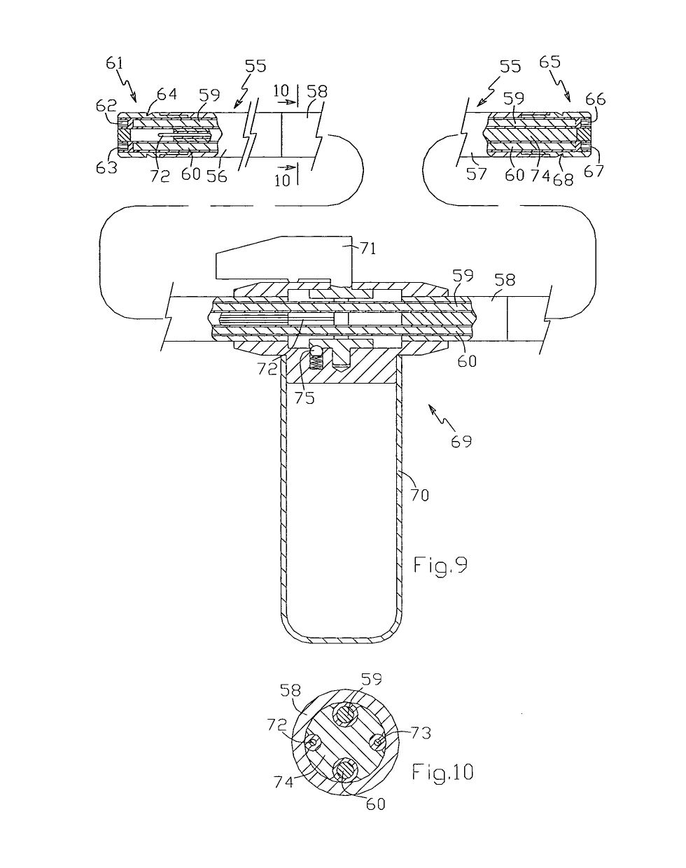

FIGS. 9, 10 show drive assembly 55 with two flexible end portions 56, 57, intermediate rigid portion 58, two drive members 59, 60, distal separable coupling means 61 with its members 62, 63, 64, as well as proximal separable coupling means 65 with its members 66, 67, 68. Two drive members 59, 60 are flexible, axially rotatable and capable of transmitting a torque from the power unit into the surgical assembly, where the rotation motion of the drive members is transformed into another needed motion kind. In this case, the power unit has two electric motors, i.e. one electric motor for every drive member. Drive assembly 55 is provided with holding means 69 disposed distally of proximal separable coupling means 65, inseparably mounted on drive assembly rigid portion 58, and including handle 70 as well as turning steering member 71. The latter is connected with flexible distal end portion 56 of drive assembly 55 by two steering wires 72, 73 and designed for turning the surgical assembly by surgeon. Drive members 59, 60 and steering wires 72, 73 are passed within drive assembly 55 and are guided with inner elastic filler 74. Steering member 71 has hermetical and movable connection with holding means 69 and is provided with underspringed fixation member 75 for the fixation of the steering member positions. Steering wires 72, 73 pass only through a relatively short portion of the drive assembly between holding means 69 and flexible distal end portion 56. This allows reducing the steering means cost, minimizing the action of the steering wires on the drive assembly operation properties, and providing the drive assembly design detachable from the power unit without eliminating the steering means.

Obviously, numerous other variations and modifications can be made without departing from the spirit of the present invention. Therefore, it should be clearly understood that the forms of the present invention described above and shown in the figures of the accompanying drawings are illustrative only and are not intended to limit the scope of the present invention.

What is claimed is:

1. A surgical apparatus, having
a surgical assembly,
a power unit providing the needed power for actuating said surgical assembly,
a drive assembly connecting said power unit and said surgical assembly, being flexible at least in the limits of the part of its length, and including
an elongated body,
and at least of one drive member designed for transmitting a driving motion from said power unit to said surgical assembly and movably disposed within said elongated body,
a distal coupling means for coupling the distal ends of said drive assembly to said surgical assembly,
a separable proximal coupling means for detachable coupling the proximal ends of said drive assembly to said power unit,
an operating means designed for an operation control of said surgical assembly and disposed on said drive assembly distally of said separable proximal coupling means.

2. The surgical apparatus of claim 1, wherein said distal coupling means is separable allowing detachable coupling the distal end of said drive assembly to said surgical assembly, and said distal and proximal coupling means have identical design allowing the use of any end of said drive assembly for coupling to said power unit and correspondently to said surgical assembly.

3. The surgical apparatus of claim 1, wherein said operating means has a stiffening means including a rigid element participating in the transmission of surgeon controlling efforts directed to said surgical assembly.

4. The surgical apparatus of claim 3, wherein said rigid element is made as a rigid portion of said drive assembly.

5. The surgical apparatus of claim 1, wherein said operating means is detachably set on said drive assembly by means of a mounting means.

6. The surgical apparatus of claim 5, wherein said mounting means includes a resilient member like a rubber spacer compressed between a clamping nut and a base of said operating means and forced out in the direction of said drive assembly thereby fixing said base on said drive assembly.

7. The surgical apparatus of claims 3, wherein said stiffening means is made as a supporting means having said rigid element detachably set on said flexible portion of drive assembly to impart a rigidity to said flexible portion of drive assembly during the surgical apparatus operation and there is a mounting means for said detachably seting.

8. The surgical apparatus of claim 1, wherein said operating means is provided with a holding means including a handle designed for holding the surgical apparatus by surgeon during the execution of the surgical operations.

9. The surgical apparatus of claim 8, wherein said operating means includes an operation desk disposed on said holding means and designed for the surgeon control of the surgical assembly motions including at least the control of said power unit.

10. The surgical apparatus of claim 1, wherein a distal end portion of said drive assembly is flexible and there is at least one steering wire disposed within said drive assembly along its axis and connecting said flexible distal end portion of drive assembly with a steering member disposed distally of said proximal separable coupling means and designed for turning said surgical assembly by surgeon, there is a holding means including a handle designed for holding the surgical apparatus by surgeon during the execution of the surgical operations and said steering member is disposed on said holding means.

11. The surgical apparatus of claim 1, wherein said power unit includes an least one electric motor, said drive member is flexible, axially rotatable and capable of transmitting a torque from said electric motor into said surgical assembly, where the rotation motion of said drive member is transformed into another needed motion kinds.

12. The surgical apparatus of claim 11, wherein there are at least two said rotatable flexible drive members.

13. The surgical apparatus of claim 1, wherein said drive member is capable of transmitting a pulling/pushing effort from said power unit into said surgical assembly.

14. A surgical apparatus, having
a surgical assembly,
a power unit providing the needed power for actuating said surgical assembly,
a drive assembly connecting said power unit and said surgical assembly, being flexible at least in the limits of the part of its length, and including
an elongated body,
and at least of one drive member designed for transmitting a driving motion from said power unit to said surgical assembly and movably disposed within said elongated body,
a distal coupling means for coupling the distal ends of said drive assembly to said surgical assembly,
a separable proximal coupling means for detachable coupling the proximal ends of said drive assembly to said power unit,
an operating means including a stiffening means designed for improving an operation control of said surgical assembly by surgeon and disposed on said drive assembly distally of said separable proximal coupling means and having a rigid element participating in the transmission of surgeon controlling efforts directed to said surgical assembly.

15. The surgical apparatus of claim 14, wherein said rigid element is made as a rigid portion of said drive assembly.

16. The surgical apparatus of claim 14, wherein said stiffening means is made as a detachable supporting means having said rigid element set on said flexible portion of drive assembly by means of a mounting means to impart a rigidity of said flexible portion of drive assembly during the surgical apparatus operation.

17. A surgical apparatus, having
a surgical assembly,
a power unit providing the needed power for actuating said surgical assembly,
a drive assembly connecting said power unit and said surgical assembly, being flexible at least in the limits of the part of its length, and including
an elongated body,
and at least of one drive member designed for transmitting a driving motion from said power unit to said surgical assembly and movably disposed within said elongated body,
a distal coupling means for coupling the distal ends of said drive assembly to said surgical assembly,
a separable proximal coupling means for detachable coupling the proximal ends of said drive assembly to said power unit,
an operating means including a controlling means designed for the surgeon control of the surgical assembly motions including at least the control of said power unit, disposed on said drive assembly distally of said separable proximal coupling means, and comprising a handle designed for holding the surgical apparatus by surgeon during the execution of the surgical operations.

18. The surgical apparatus of claim 17, wherein said controlling means includes an operation desk disposed on said handle and communicated with said power unit.

19. The surgical apparatus of claim 17, wherein said controlling means comprises a steering member designed for turning said surgical assembly by surgeon through at least one steering wire disposed within said drive assembly along its axis and connecting a flexible distal end portion of said drive assembly with said steering member.

20. The surgical apparatus of claim 17, wherein said controlling means has a stiffening means including a rigid element participating in the transmission of surgeon controlling efforts directed to said surgical assembly, therewith said handle is located in the limits of said rigid element.
